(19) United States
(12) Patent Application Publication
(10) Pub. No.: US 2019/0183185 A1
(43) Pub. Date: Jun. 20, 2019

(54) A SYSTEM AND METHOD OF MONITORING AND CONTROLLING THE USAGE BEHAVIOUR OF AN ELECTRONIC CIGARETTE

(71) Applicant: DR. REDDY'S LABORATORIES LIMITED, Hyderabad (IN)

(72) Inventors: Mukund Karambelkar MANAS, Jalgaon (IN); Chakravarty SHREYA, New Delhi (IN)

(21) Appl. No.: 16/322,700

(22) PCT Filed: Aug. 3, 2017
(86) PCT No.: PCT/IB2017/054757

§ 371 Date: Feb. 1, 2019

(30) Foreign Application Priority Data
Aug. 3, 2016 (IN) 201641026438

(51) Int. Cl. A24F 47/00; A24F 7/00; A24F 21/00; A24B 15/16; A61M 15/06

(52) U.S. Cl. CPC A24F 47/008; A24F 7/00; A24F 21/00; A24B 15/167; A61M 15/06; A61M 2205/8206; A61M 2205/3584; A61M 2205/3653; A61M 2205/582; A61M 2205/583; A61M 2205/3569

(57) ABSTRACT

The present invention relates to a system and method of monitoring and controlling the usage of an electronic cigarette which helps the user gain control over their habit by making them aware of their habit, setting tangible goals, creating personalized plans and ensuring adherence. The usage behavior is communicated to the user by displaying on the body of the electronic cigarette; information such as percentage of current conventional tobacco based cigarette being smoked, total number of conventional tobacco based cigarettes smoked in a day etc. This usage behavior is calculated by correlating the amount of vapor inhaled in one sitting from electronic cigarette to be equivalent to the smoke inhaled from usage of one conventional tobacco based cigarette. The systems and methods described herein further utilize a electronic cigarette in combination with a mobile-device-based application on a secondary processing unit such as mobile device or remote personal computer system to track and control the usage behavior of consumption of electronic cigarette—through the Track, Reduce or the Quit Cold Turkey approaches. The user can control the usage of electronic cigarette by setting a target date as well as create a personalized plan for gradual reduction in smoking. The system further provides the user flexibility by allowing the user to limit the consumption of electronic cigarette, eventually leading to quitting smoking.

### CROSS-REFERENCE TO RELATED APPLICATIONS

This application claims the benefit of Indian Provisional Application No. 201641026438, filed Aug. 3, 2016, which application is incorporated herein by reference in its entirety.

### FIELD OF THE INVENTION

The present invention generally relates to a nicotine delivery system. More particularly, the present invention relates to system and method of monitoring and controlling the usage of an electronic cigarette in a nicotine delivery system by a user.

### BACKGROUND OF THE INVENTION

It is usually seen that the individuals (user) who are habituated with the tobacco based smoking articles such as cigarette and cigars for a long time, tend to lean toward serious health conditions. The smoke produced by the tobacco based smoking articles such as cigarette or cigar contains extremely harmful substances such as tar. Cigarette Smoking is recognized as a major health risk. In United States alone, on an average, 435.000 people die prematurely from smoking-related diseases each year.

A genuine attempt of quitting smoking using a ‘Cold Turkey’ method i.e. stopping smoking immediately, results in severe withdrawal symptoms with a high chance of relapse. The addiction is primarily due to nicotine. This warrants a lot of willpower at the individual level to free themselves from nicotine addiction. Additionally, abrupt quitting of smoking deprives the individual of the enjoyment particularly, of the flavor of tobacco products derived from smoking. This loss of enjoyment is a substantial factor for individuals seeking to quit smoking.

Nicotine is a neutral chemical which attaches to specific cell receptor sites in the brain. Therefore, preventing its ability to attach to these cell receptor sites is one of the methods used to interfere with the addictive qualities of nicotine. One such alternative treatment involves the use of chemical compounds which bind to these cell receptor sites, and thereby prevent nicotine from binding to those cell receptor sites. As a result, the nicotine based cravings of the individual are reduced. Additionally, Nicotine replacement products such as chewing gums permit the individuals to satisfy the body's craving of nicotine thereby preventing the damage to lungs caused by inhalation of the tobacco smoke. Projected as one of the earlier successful approaches to reduce nicotine withdrawal symptoms, these chewing gums suffered from issues such as bad taste as well as GI disturbances. Later, nicotine containing transdermal patches were developed with the aim of reducing nicotine craving by providing nicotine through transdermal absorption at a rate which is sufficient to correspond to the nicotine level in the blood which the smoker achieves through normal smoking patterns. Interestingly, a combination of nicotine containing transdermal patch and a nicotine containing aerosol has also been disclosed in U.S. Pat. No. 5,016,652A, assigned to Rose. While the patch provides the nicotine levels in the blood with respect to smoking patterns, the aerosol aids in providing nicotine of a selective droplet size and concentration to simulate the sensations i.e. craving achieved through tobacco smoke.

A visible concern in using nicotine replacement therapies such as patches is the constant requirement of placing the patch on the skin at least for a period of 24 hours. This makes the individuals feel like they are unable to control the nicotine and cannot see how much nicotine is going into their body. The perception that people get is that nicotine patches are trying to box them in a certain category. “If you smoke more than 20 cigarettes a day then start at step 1 (21 mg)”. The nicotine levels do not match with people's habit and they still feel some withdrawal symptoms. The nicotine delivery of all day patches is also unpredictable and does not match the nicotine ‘routine’ that people have set for themselves. They hence feel uncomfortable and question if it is too much or not the amount they need. As a result, nicotine supplements solve only some of the problems associated with smoking. The current communications related to nicotine replacement products also make people feel as if they are addicts requiring some help. Users don't identify themselves as addicts and the addiction language pushes them away.

All these nicotine replacement products and attempts have the ultimate aim of reducing negative health consequences. Despite these benefits, while nicotine supplements protect the individual from the significant harm that smoking causes by satisfying the need to obtain nicotine without inhaling tobacco smoke, they do not overcome the individual's addiction to nicotine all of the time. In addition, once the chewing gum is discarded, or the transdermal patch is no longer used, the craving for tobacco products often returns.

Alternatively, there has been a proliferation of tar-free cigarettes by introducing electronic smoking articles e.g. electronic cigarettes, for eradicating major tar related health issues faced by many users who are habituated with conventional tobacco based smoking articles.

Further, since the electronic cigarettes are easy to use and may expose a user to continue an unquantified use of said smoking articles, this gives rise to a drawback of unregulated use of such nicotine based electronic smoking articles.

There is another dimension to controlling the smoking habit and that is the mindset of the user. The definition of quitting is different from one person to another; being in control of the habit is more important for users who not necessarily mean quitting smoking. Users find it very difficult to switch to nicotine patches as the language used is in milligrams and not in the number or strength of cigarettes. Some users consider staying in their set limit as their quit goal and some think of smoking within set rules as their quitting goal. This means that each user wants to be in control of their habit, and not necessarily go down to zero cigarettes.

The fear of relapse for the users is about opening themselves to public judgment and embarrassment. A gradual, easily measurable reduction in the number of cigarettes is convenient for the user. It takes the pressure of quitting all of a sudden as quitting is a huge physical and emotional commitment on the part of the user.

Furthermore, the existing arts do not have a capability of monitoring the regular usage of the smoking article. There is a possibility that users might inhale more nicotine using electronic cigarettes as compared to how much they did when they were smoking conventional tobacco based cigarettes, as the user is unable to count it in case of electronic cigarettes.

Hence, there exists a need in the art to provide a nicotine delivery system and method of monitoring the usage behavior of the electronic cigarette in a nicotine delivery system. Further, it is required to propose a mechanism to regulate the usage by the user based on the previous usage of the cigarette and/or metabolic state of the user and eventually allowing the user to quit the habit of smoking.

### OBJECTIVE OF THE INVENTION

The primary objective of the present invention is to control the consumption of an electronic smoking article in a nicotine delivery system.

The objective of the present invention is to control the consumption of an electronic cigarette in a nicotine delivery system.

Yet another objective of the present invention is to detect the user behavior of consuming the electronic cigarette.

Yet another objective of the present invention is to monitor the consumption of the electronic cigarette.

Yet another objective of the present invention is to provide visual representation of consumption of the electronic cigarette.

Yet another objective of the present invention is to control the operations of the electronic cigarette by locally changing the configuration electro-mechanically.

Yet another objective of the present invention is to control the operations of the electronic cigarette through a user device in communication with the electronic cigarette.

Yet another objective of the present invention is to control the operations of the electronic cigarette through a remote computing device centrally.

Yet another objective of the present invention is to guide the user to reduce the usage of electronic smoking article by the user.

Yet another objective of the present invention is to provide a modular electronic smoking article.

Yet another objective of the present invention is a method of tracking the usage behavior of consumption of an electronic cigarette, the method comprising: displaying on the body of the electronic cigarette, the amount of nicotine inhaled by the user during the consumption of the electronic cigarette.

Yet another objective of the present invention is a method of controlling the usage behavior of consumption of an electronic cigarette, the method comprising: enabling the user to manually set a daily consumption limit equivalent to number of conventional tobacco based cigarettes by using the set limit button on the body of the electronic cigarette; and wherein upon reaching the daily consumption limit, the electronic cigarette goes into lock mode where the user cannot use it anymore.

Yet another objective of the present invention is a method of assisting in tracking and controlling the usage behavior of consumption of an electronic cigarette, using one or more secondary processing units, the method comprising:
displaying at least one or more graphical user interfaces inviting the user to enter data describing the user's current smoking habit,
receiving data from the user, the data being indicative of the user's current smoking habit,
displaying at least one or more graphical user interfaces inviting the user to select either a tracking or a controlling approach,
receiving data from the user, the data being indicative of the selected approach.

Yet another objective of the present invention is a nicotine delivery system comprising:
a) an electronic cigarette comprising:
   a mouthpiece (009),
   a cartridge (015),
   an airflow and vapor sensor (008),
   a heating coil (011),
   a set limit button (005),
   a capacitive sensor (003),
   a main power circuit board (002),
   a battery (013),
   a Light Emitting Diode display (001), and
b) an electronic cigarette holder which holds the electronic cigarette when it is not in use.

### BRIEF DESCRIPTION OF THE DRAWINGS

FIG. 1a is a cross-sectional view of the electronic cigarette in a nicotine delivery system of the present invention

FIG. 1b is a top view of the electronic cigarette in a nicotine delivery system of the present invention

FIG. 2a illustrates the work flow diagram depicting the functioning of the electronic cigarette in a nicotine delivery system.

FIG. 2b is a network diagram including the electronic cigarette in a nicotine delivery system and the secondary processing units.

FIG. 2c is a network diagram of the different components of the CPU Block FIG. 3 is a network diagram of the different components of the electronic cigarette holder in a nicotine delivery system.

FIG. 4 illustrates a flow diagram of the method of monitoring the usage consumption of the electronic cigarette as per one embodiment of the invention.

FIG. 5 illustrates the different approaches and the personalized plan the user may opt in order to monitor and control the usage consumption of electronic cigarette.

FIG. 6a illustrates the graphical user interface of a mobile-device-based application installed on a secondary processing unit and created as per one embodiment of the present invention which provides the user the options to select one of the approaches to monitor and control the usage consumption of the electronic cigarette.

FIG. 6b illustrates the habit pattern of the consumption of electronic cigarette equivalent to number of conventional tobacco based cigarettes smoked at different locations and monitored through a graphical user interface of a mobile-device-based application installed on a secondary processing unit and created as per one embodiment of the present invention.

FIG. 6c illustrates the visualization of the number of conventional tobacco cigarettes smoked and urges overcome against the current plan created through a graphical user interface of a mobile-device-based application installed on a secondary processing unit and created as per one embodiment of the present invention.

FIG. 6d illustrates the plan creation screen for the “Reduce” or “Slow Down” approach on the graphical user interface of a mobile-device-based application installed on a secondary processing unit, which allows the user to set a target or goal date or duration in terms of number of weeks within which the user intends to reduce to “0” number of cigarettes.

### DETAILED DESCRIPTION OF THE INVENTION

The present invention encompasses a system and method of monitoring and controlling the usage of an electronic cigarette in a nicotine delivery system by a user, wherein the e-cigarettes are a type of compact aerosol delivery device which produces vapor upon dragging/inhaling air from one end.

As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel it is believed that the invention will be better understood from a consideration of the following description in conjunction with the drawing figures, in which like reference numerals are carried forward

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure.

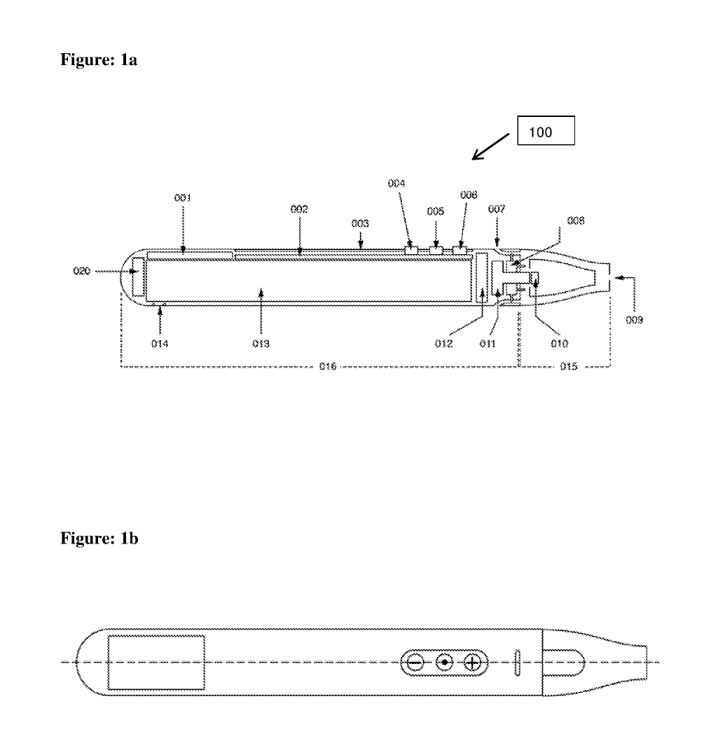

Referring now to the drawings in detail and first, particularly to FIG. 1a, there is shown a first exemplary embodiment of a nicotine delivery system comprising an electronic cigarette and an electronic cigarette holder which holds the electronic cigarette when it is not in use.

Electronic cigarette (100) is made up of a metal body and comprises of a
a mouthpiece (009),
a cartridge (015) containing nicotine liquid along with a wick (010),
an airflow and vapor sensor (008),
a heating coil (011),
an air vent (007),
a relay (012),
a body (016),
a plus button (006),
a set limit button (005),
a minus button (004),
a capacitive sensor (003),
a main PCB (002) (which comprises of micro-controller, thermistor and wireless transceiver),
a battery (013),
an LED display (001),
a charging terminal (014),
a vibration motor (020),

The cartridge (015) is a small plastic see through container. It contains the nicotine liquid concentrations which may be of various concentrations/strengths and with or without flavors to match the nicotine levels and flavors in the conventional tobacco based cigarettes for e.g. the amount of nicotine in the nicotine liquid may be selected from about 0.6 mg, 1.1 mg, 1.6 mg or 2.4 mg. According to certain embodiments, it may also include a non-nicotine liquid for e.g. a mixture of propylene glycol, glycerin and one or more flavors. This eventually makes it easier for the user to switch to e-cigarette as it would match their current smoking habit. Some example of flavors that can be delivered using the cartridge are menthol, chocolate, vanilla, coffee, tobacco, cherry, etc. The cartridge has a wick (010) at one of its end which is soaked by the nicotine liquid and comes in contact with the heating coil (011) when attached to the body and other end open for user as mouthpiece (009).

The nicotine cartridge itself is detachably connected to the heating coil (011). It may be removed and replaced with another cartridge when the nicotine liquid is consumed. The user is able to visualize the level of the nicotine liquid left in the cartridge.

Each cartridge further comprises of a chip which acts as a unique Identifier (UID) allowing the primary processing device of the electronic cigarette to detect the type of cartridge (strength of nicotine and/or a non-nicotine cartridge). Once the original cartridge is used to the prescribed amount of time, the cartridge becomes void. This prevents the user from using low quality counterfeit cartridges or manually refilling the cartridge with a subpar nicotine infused liquid.

The air vent (007) provides the inlet for passage of air to enable the user to drag on the mouthpiece (009).

The airflow and nicotine vapor sensor (008) measures the change in pressure when the user drags on the mouthpiece (009) to activate the microcontroller and start the heating coil (011). The other function of the sensor is to measure the volume of the vapor being generated that may be used to measure the actual amount of nicotine being inhaled. This method of measuring the usage is more accurate since there may be some amount of loss between the volume of liquid in the cartridge and the volume of the vapor being generated.

The function of the heating coil (011) is to heat up the liquid soaked in the wick.

The function of relay (012) is to control the flow of current from the battery (013) of the electronic cigarette (100) to the heating coil (011).

The main power circuit board (PCB) comprises of the micro-controller, thermistor and the wireless radio transceiver.

The micro-controller runs the software on the electronic cigarette.

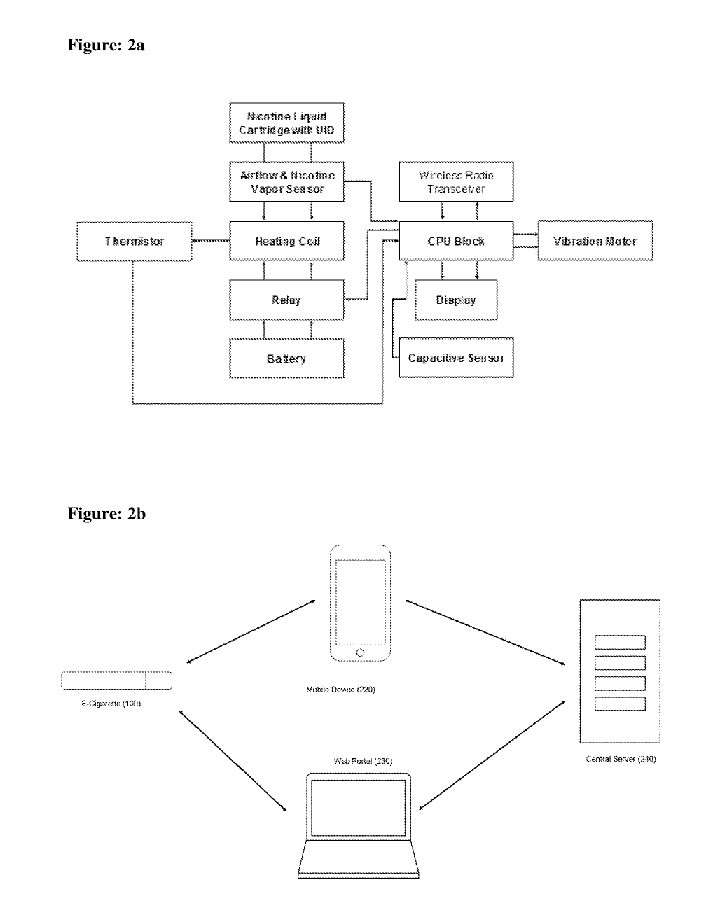

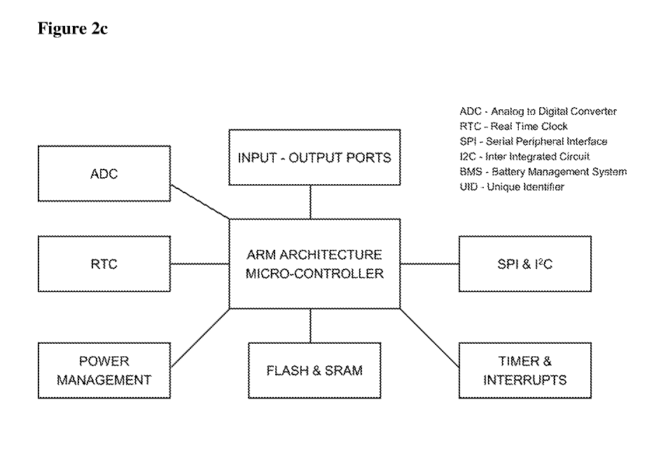

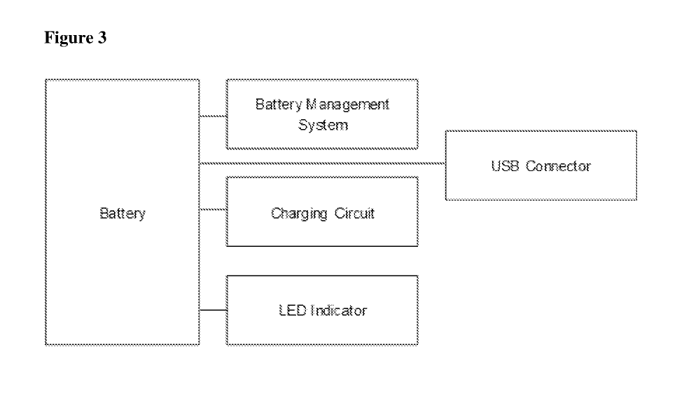

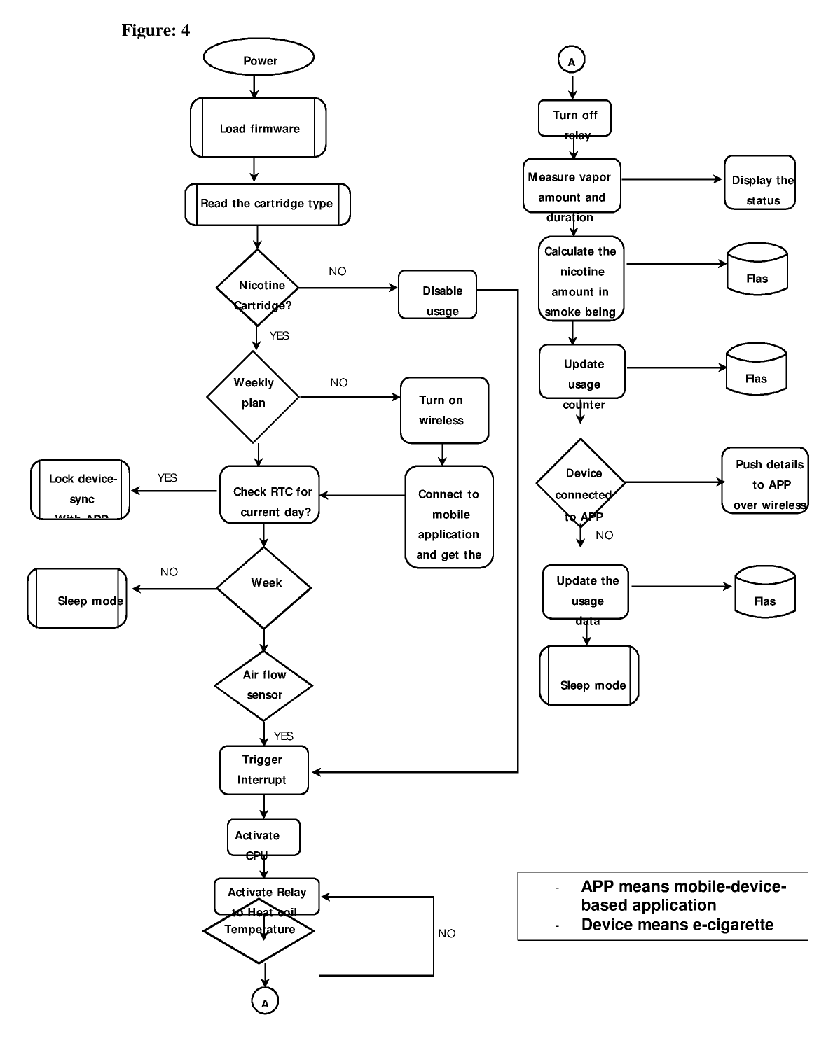

The function of the thermistor (018) is to check the temperature of the heating coil (011) and transmit it to the Central Processing Unit (CPU) (FIG. 2a).

The function of the wireless radio transceiver is to connect wirelessly to mobile-device-based application on the secondary processing unit or a remote personal computer system to receive the user's plan data and send the electronic cigarette usage data using any form of short range communication means such as infrared, Bluetooth, etc.

The battery (013) in the electronic cigarette (100) may be lithium-ion rechargeable battery and is housed inside a shell. The battery provides power for the heating coil (011) to generate vapor and to power the entire circuitry for it to function.

A Light Emitting Diode (LED) display (001) or any suitable lighting source informs the user about the various states regarding the usage, battery level and syncing of the electronic cigarette. For example, the LED display is configured near the battery. The display remains invisible when the device is not in use. The LED display of the electronic cigarette or the device is digitally drawn with a graphical pattern which moves across the length of the electronic cigarette at the far end having battery and moves toward the cartridge end having the mouth piece portion. The swiftness of moving of the pattern may be proportional to the flow of the vapor delivered to the user every time the user drags. The pattern reaches the cartridge end when the user inhales amount of nicotine through vapor which is equivalent to the amount of nicotine inhaled through smoke from one conventional tobacco based cigarette which may be further configured as per the requirements. The pattern may start displaying from the far end when the user continues dragging after consumption of previous portion of the electronic cigarette and accordingly, the count may be taken as a ‘next cigarette’. The display is provided with current from the battery for a predefined ideal mode for e.g. the current may be delivered to the display and other components of the electronic cigarette until two minutes, if the user does not drag the device in between. Therefore, the system may reset the display representation of the consumption of the electronic cigarette and start as a new cigarette when next time the user drags the electronic cigarette.

The charging terminal (014) aids in charging the electronic cigarette using the electronic cigarette holder. The electronic cigarette may also be charged with other attachments or cables wherein user does not necessarily need to have the device holder.

The capacitive sensor (003) senses any conductive object such as a tap action by the human finger and informs the CPU block to display the usage data.

The set limit button (005) on the body (016) of the electronic cigarette allows the user to manually set a daily consumption limit of electronic cigarette equivalent to number of conventional tobacco based cigarettes using the plus (006) and minus button (004).

The vibration motor (020) at the opposite end of the mouthpiece provides a light vibration to indicate parameters such as end of personal daily limit, battery low etc.

The exterior mechanical enclosure of electronic cigarette may change in diameter, length, or material based on industry appearance requirements. These requirements may include branding, tip color, filter color, metal finish, etc.

FIG. 2a illustrates the work flow diagram depicting the mechanical/electronic functioning of the electronic cigarette. As the user drags on the mouthpiece, the airflow and vapor sensor measures the change in pressure and activates the microcontroller and starts heating the coil. The heating coil draws current from the battery through the relay for converting the nicotine liquid absorbed in the wick of the cartridge and converts it into a vapor to be inhaled by the user at a preferably constant rate of nicotine flow, for example 0.02 mg per second. The vapor is released back into the mouth of the user by avoiding direct release of liquid into the mouth of the user. The rate of vaporisation depends on the electrical resistance of the heating coil, the current drawn by the heating coil and the duration for which the heating coil is operated. Once the temperature required to convert nicotine liquid to vapor has reached, the thermistor informs the CPU block of the temperature which then receives the volume of the vapor being generated from the airflow and vapor sensor and then the actual amount of nicotine being inhaled is measured. The CPU block (FIG. 2a) is the primary processing unit which controls the device. This measured amount is displayed through a LED display on the device and concurrently sends through the wireless radio transceiver to the secondary processing unit.

In FIG. 2b, a network diagram including the electronic cigarette and the secondary processing units as per one embodiment of the invention is illustrated. The diagram represents information flow between the electronic cigarette and the secondary processing units. The system comprises an electronic cigarette 100 for consumption by the user; a mobile-device-based application or the app installed on a secondary processing unit such as mobile device for e.g. a smart phone 220 in communication with the primary processing unit of the electronic cigarette 100 for recording and storing data associated with the electronic cigarette 100. Alternatively, the primary processing unit of the electronic cigarette 100 is in communication with another secondary processing unit such as remote personal computer system 230 for recording and storing data associated with the electronic cigarette 100. The data can be accessed by the user by logging through a web portal on the remote personal computer system 230. The data recorded on either the mobile device 220 or the remote personal computer system 230 can further be stored on the remote cloud-based application or the central server 240. The mobile device or the remote personal computer system communicates through the remote cloud-based application or the central server through the global system of interconnected computer networks or the Internet.

FIG. 2c is a network diagram of the different components of the CPU Block present in the electronic cigarette. The ARM Architecture Micro-controller is at the heart of the CPU which processes all the data. The Analog to Digital Converter (ADC) converts all the analog data inputs such as the volume of vapor, temperature of the heating coil into digital input for the micro-controller to process it. All the components of the CPU and the device are connected at the Input Output Ports of the micro-controller to send and receive data. The Real Time Clock (RTC) lets the microcontroller know the current date and time which is tagged to every usage session of the device. The Power Management manages the power for the CPU block. The Flash & SRAM is the on-device data storage to store the weekly plan which contains the daily limits for device usage. It also stores the usage data. The Timer & Interrupts are the components that wake the micro-controller when the user tries to use the device and put the device in sleep mode when not being used to conserve the battery. The SPI & I2C are the communication protocols which aid in communication between the integrated circuits in the CPU Block.

FIG. 3 is a network diagram of the different components of the electronic cigarette holder in a nicotine delivery system which holds the electronic cigarette when it is not in use. It also includes the spare cartridges of various or same strength and flavor. The holder also functions like a portable charger for the device. This helps in keeping the battery size in the electronic cigarette itself small so that it matches the size of the conventional tobacco based cigarette or electronic cigarette making it easier to hold and use. The holder has a lithium-ion battery which is charged using a USB connector. The charging circuit controls the flow from the USB charger and the Battery Management System manages the charging and discharging of the battery and the electronic cigarette. An LED indicator is used to notify the user about the battery level in the electronic cigarette holder. When the electronic cigarette is stored in the electronic cigarette holder, the connector pins at the charging terminal of the electronic cigarette meet the corresponding charging pins of the holder. This starts the charging of the device battery. The electronic cigarette is secured inside the holder using magnets or snap mechanism. The electronic cigarette is always stored in the device holder which functions like a portable charger for the device. It also keeps the device hygienic by avoiding the contact of the mouthpiece with other objects.

FIG. 4 illustrates a flow diagram depicting the method of monitoring the usage consumption of the electronic cigarette as per one embodiment of the invention. As soon as the user pulls the electronic cigarette out of the electronic cigarette holder, the electronic cigarette is turned on. It loads the firmware in memory and identifies the type of cartridge currently installed in the electronic cigarette. If the cartridge does not contain any nicotine and is not used up, the electronic cigarette will jump into the usage and tracking mode since user can use the non-nicotine cartridge without any limits.

If the cartridge contains nicotine, the device will check if the weekly plan is created and stored on the device. If it is not stored on the device, it will connect to the mobile-device-based application on the secondary processing unit such as mobile device wirelessly and download the plan on the device memory. If it already has a weekly plan stored, it will check the Real-Time Clock (RTC) on the device to see if the week for which the plan is stored is already complete. If the week for which the plan was stored is already complete, the device will try to sync with the mobile-device-based application and download the weekly plan for the current week. Once the current week plan is loaded on the device, the device will wait for the user to drag on to the mouthpiece of the device. This will activate the airflow and vapor sensor and will trigger the interrupt awake pin to activate the CPU. If user does not use the device after pulling it out of the holder for certain duration, the device will automatically fall into sleep mode to conserve battery.

Once the user starts dragging on the mouthpiece and the CPU is activated, it will in turn activate the relay to start heating up the heating coil. The thermistor will keep checking the temperature of the heating coil for the right temperature of the nicotine liquid evaporation. Once the right temperature is reached, the relay will be turned off to cutoff the power supply from battery to the heating coil avoiding the overheating of the coil and additionally a burnt taste for the user.

As the nicotine liquid turns into vapor and escapes the device into the user's mouth, the volume of the vapor is measured to calculate the amount of nicotine being inhaled by the user. The display imitates the visual feedback same as that of a conventional tobacco based cigarette which provides to a smoker by burning off.

The display shows the amount of nicotine inhaled by the user in the form of percentage of the amount of nicotine from a single conventional tobacco based cigarette. This is calculated based on the volume of vapor being generated in comparison to the volume of smoke produced by a single conventional tobacco based cigarette. Once the 100% volume of nicotine vapor equivalent to a conventional tobacco based cigarette is inhaled by the user, the device displays the amount of nicotine inhaled by the user in the form of a number; wherein the number is equivalent to the number of conventional tobacco based cigarettes. The user may continue using the electronic cigarette and the percentage usage would be displayed for the next conventional tobacco based cigarette. In one embodiment, the count for a day may be 24 hours of time period from 6 am of a calendar day to 6 am of the next calendar day. The percentage or total count of cigarettes resets to zero on completion of the 24 hour time period.

When user is not using the device, the user is enabled to see the usage consumption amount anytime by tapping the device body which has a capacitive sensor (003). The sensor in turn activates the display.

The first tap on the device body displays total count of the number equivalent of conventional tobacco based cigarettes smoked on that particular day. While this number is being displayed if the user taps again on the device body, the display shows the battery percentage of the device in visual form.

On completion of the amount of nicotine vapor equivalent to a single conventional tobacco based cigarette, the device checks for wireless connectivity with the mobile-device-based application on the mobile device. If the mobile-device-based application and the device are already connected, the device pushes the usage data to the app. The app then tags the cigarette consumed recently with the location information received from the mobile device, either using GPS (Global Positioning System) data or Network (WiFi access points & Cellular tower) data. The app may also ask the user to provide additional subjective information about the cigarette such as what triggered them to smoke and how were they feeling when they smoked.

The objective (time, number and location) and subjective (trigger, emotion) data of every cigarette equivalent to the smoke inhaled from a conventional tobacco based cigarette forms a habit pattern over time. The user is enabled to gain insight into their habit which could help them gain control over it by knowing what makes them smoke more and then avoiding those situations or being more mindful of those situations.

While using the device to inhale the vapor, if the total count of the cigarettes smoked has reached the limit of that day's limit of number of cigarettes, the device goes into lock mode where user cannot use it any more. User is notified about this with a light vibration displaying a ‘X’ symbol on the device display to indicate that the limit has been reached. Users may use a non-nicotine cartridge if they wish to smoke beyond the limit established in the plan. The non-nicotine cigarettes are counted and stored differently and they do not add into the daily number to exceed the limit. The light vibration is initiated using a vibration motor (020) which is connected to the CPU in the device.

The device can store the usage data up to 7 days in addition to the weekly plan data. This reduces the dependency of the app being always connected to the device for the intended use of controlled smoking.

The usage data is stored in the device memory tagged with the date and time information from the RTC. Further, the data may be stored at the remote personal computer system 230. This enables the user to create personal accounts as well as share progress with social connections as well save progress on a remote cloud-based application 240.

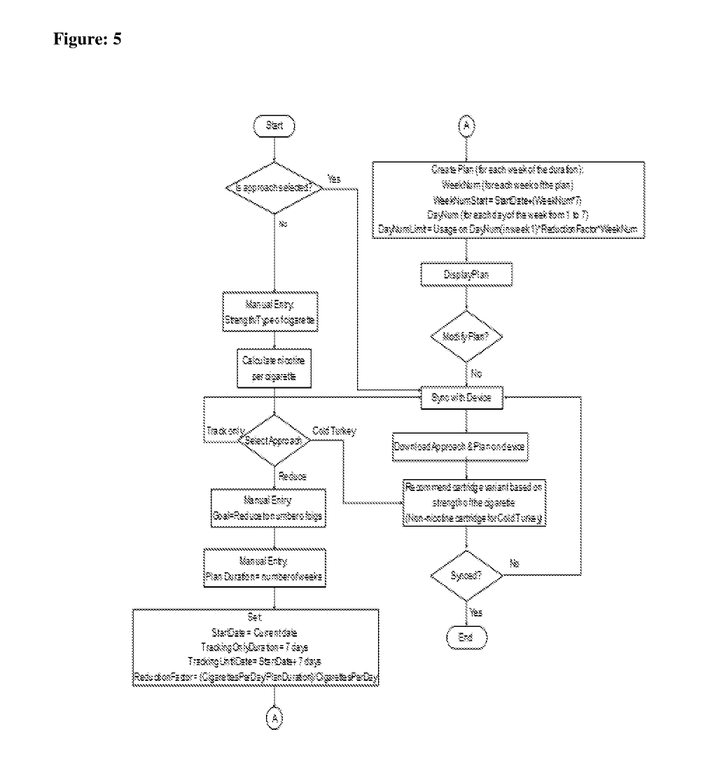

FIG. 5 illustrates the different approaches and the personalized plan the user may opt to monitor and control the usage consumption of electronic cigarette.

As part of the initial setup on the mobile device app, the users are asked few questions to gather information on their smoking habit to detect addiction level and motivation to quit. Users are also asked for the type of conventional tobacco based cigarettes they smoke.

Based on this information, the appropriate variant of the cartridge is recommended to match the nicotine level they are inhaling. This itself cuts down on the tar and other harmful chemicals they are inhaling without making any significant difference to their habit. The types of conventional tobacco based cigarettes may be “Light” (containing about 0.6 mg nicotine) or “Medium” (containing about 1.1 mg nicotine) or “Strong” (containing about 1.6 mg nicotine) or “Extra Strong” (containing about 2.4 mg nicotine). Accordingly, a 6 mg cartridge (equivalent to 10 conventional tobacco based cigarettes) is available to the user as a cartridge variant if he wants to use the “Light” type of cigarette. A 11 mg cartridge (equivalent to 10 conventional tobacco based cigarettes) is available to the user as a cartridge variant if he wants to use the “Medium” type of cigarette. A 16 mg cartridge (equivalent to 10 conventional tobacco based cigarettes) is available to the user as a cartridge variant if he wants to use the “Strong” type of cigarette. A 24 mg cartridge (equivalent to 10 conventional tobacco based cigarettes) is available to the user as a cartridge variant if he wants to use the “Extra Strong” type of cigarette.

User is then asked to select one of the approaches they would like to take to gain control over their habit. The options are “Just Track” (or “Watch the Road”) with which the user can track their smoking habit using the electronic cigarette.

Users do not set any quitting goal or plan but being aware of the habit itself could bring change in their habit. This is further explained in next section of habit pattern.

Alternatively the user can opt for “Quit Cold Turkey” (or “Hit the Breaks”) approach if they want to quit smoking immediately. They are recommended to use the non-nicotine cartridge in their electronic cigarette. This is so that even if they can manage the nicotine cravings it might be hard to give up the habit of hand and mouth engagement that the conventional tobacco based cigarettes provide along with the opportunity to take a break, have a moment alone and socialize with other smokers. In these situations of weakness, there is a high probability of users to relapse into smoking, this approach enables the user to use the non-nicotine cartridge and have the same experience of smoking without inhaling any addictive nicotine.

Another suitable approach is “Reduce” (or “Slow Down”). In this approach, the users enter the number of conventional tobacco based cigarettes they smoke currently and the number of conventional tobacco based cigarettes that they want to reduce to. They also select the duration in which they want to achieve their goal. The duration is ideally set in number of weeks ranging from 2-8 weeks but could be longer than 8 weeks as well.

Based on the approach selected, the app creates a plan (for reduction). The first week of the plan is where users smoke as they usually do without reducing the number of conventional tobacco based cigarettes they do using the equivalent nicotine vapor amount form the electronic cigarette and the recommended or selected cartridge strength. The consecutive weeks of the plan reduce the number of conventional tobacco based cigarettes a user can smoke in linear fashion to their current habit. For example, If a user currently on an average smokes 12 conventional tobacco based cigarettes in one day, and set a goal of reducing to 0 in 4 weeks. This would mean that from the 5^{th} week onwards, the user would not be smoking anymore. Accordingly, the weekly average reduction factor is calculated using the formula:

Reduction Factor=Average conventional tobacco based cigarettes per day/Duration of plan

In this example, Reduction Factor will be 3. Based on this average reduction factor, Weekly limits (Week Num Limit) are set using the formula:

WeekNumLimit=Average cigarettes per day−[(WeekNum−1)*Reduction Factor]

“Week 1” will involve the tracking of the habit using the electronic cigarette only to arrive at baseline value. The amount of nicotine vapor to be inhaled equivalent to average number of conventional tobacco based cigarettes to be smoked will reduce to 9 calculated based on the reduction i.e. “12−3=9”.

Similarly, it will be a total of 6, 3 and 0 respectively for “Week 3”, “Week 4” and “Week 5”. The Week Num Limit i.e. Week 1 Limit, Week 2 Limit, Week 3 limit will be variable for each of the weeks.

This personalized plan is displayed on the mobile device app which the user can choose to modify to their liking. They can add or remove weeks to the plan duration and change the weekly limit number. Once the plan is created and confirmed by users, they are prompted to sync the electronic cigarette or the device with the mobile device app and are informed to install the recommended cartridge in the electronic cigarette. Upon syncing with device over wireless radio transceiver, the electronic cigarette stores the information such as selected approach, plan start date, tracking start date, tracking end date, and week 1 plan. Once the device is synced with the app, it indicates the same to user by displaying it on the device display and a light vibration.

Additionally, the users may choose to secure the device by software lock. This can be done by setting a unique pin or a password or pattern lock or using the fingerprint sensor (if available on the mobile device). This makes the device more secure as it prevents it from being used by other people or minors who are not legally allowed to use such electronic cigarettes.

The first week of the plan is where the daily smoking habit is tracked to establish a baseline habit pattern. This lets the app know about how the habit differ on all the 7 days of the week. For example, if the average number of conventional tobacco based cigarettes smoked approximates at 12 cigarettes per day i.e. starting with first day of the week say Sunday, the user smokes 9 conventional tobacco based cigarettes, second day of the week i.e. Monday the user smokes 8 conventional tobacco based cigarettes. Similarly the number of conventional tobacco based cigarettes smoked by the user are 10, 8, 9, 17 and 20 respectively for the next 5 days. With the week 2 plan already set to average 9 conventional tobacco based cigarettes per day; the mobile device app based on the habit pattern of the user, instead of applying the limit of 9 cigarettes on all 7 days, it reduces the number proportionately for each day, i.e. limits of week 2 would then be on first day of the second week i.e. Sunday, the user will be only allowed to amount of nicotine vapor equivalent to 6 (i.e. 9-3) conventional tobacco based cigarettes. Similarly the nicotine vapor amount equivalent to the number of conventional tobacco based cigarettes allowed to be smoked by the user will then equate to 5 (i.e. 8-3), 7 (i.e. 10-3), 5 (i.e. 8-3), 6 (i.e. 9-3), 14 (i.e. 17-3) and 17 (i.e. 20-3) each for the next 6 days. This daily reduction will enable to achieve the average weekly limit of approximately to 9 cigarettes per day. This method of reduction is more tuned to users' current habit which makes it easier to gain control over their habit. The plan continues in similar fashion until user has reached 0 cigarettes per day. After which user might choose to use the non-nicotine cartridge with the electronic cigarette to overcome cravings without relapsing into nicotine addiction.

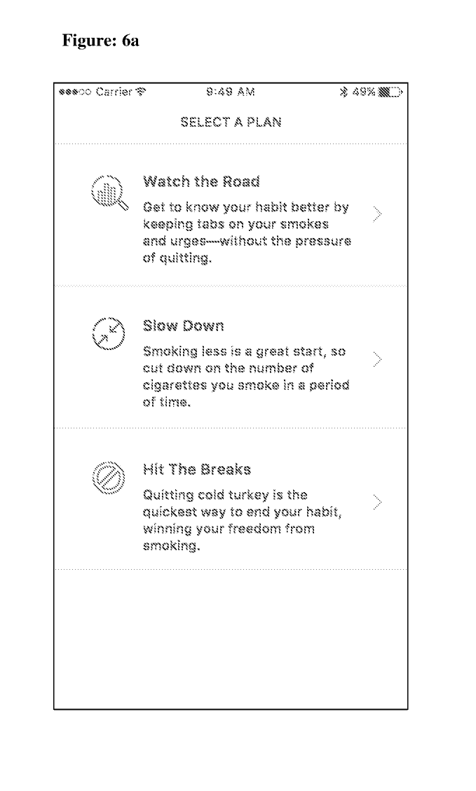

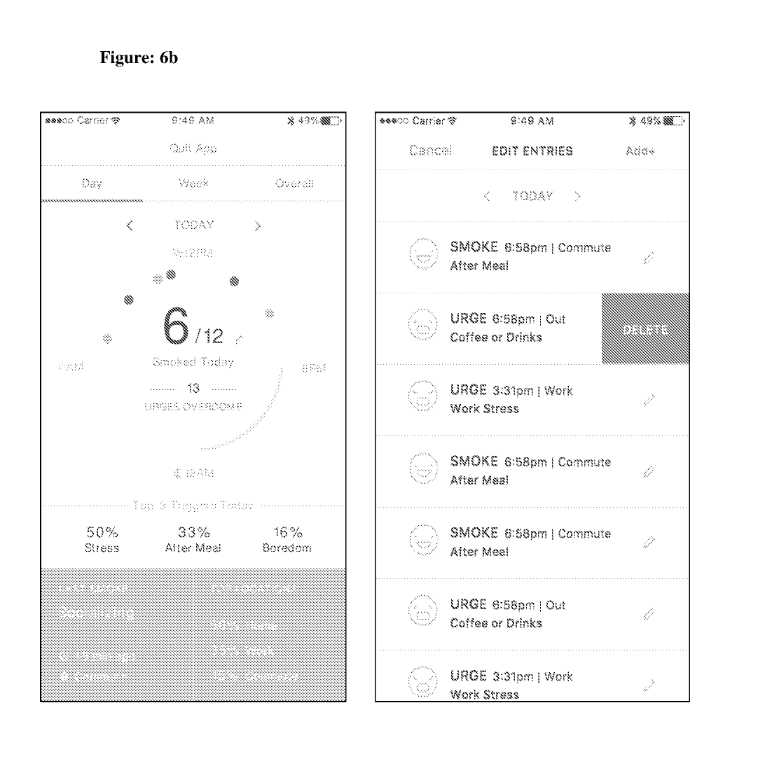

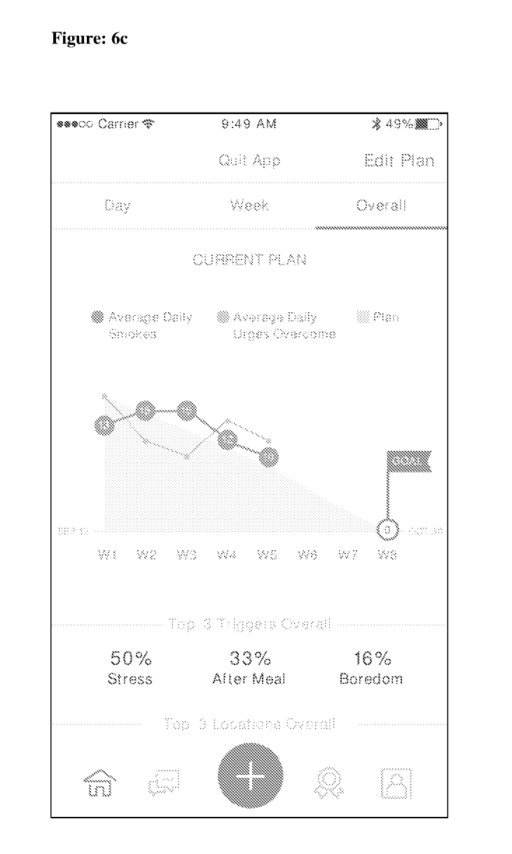

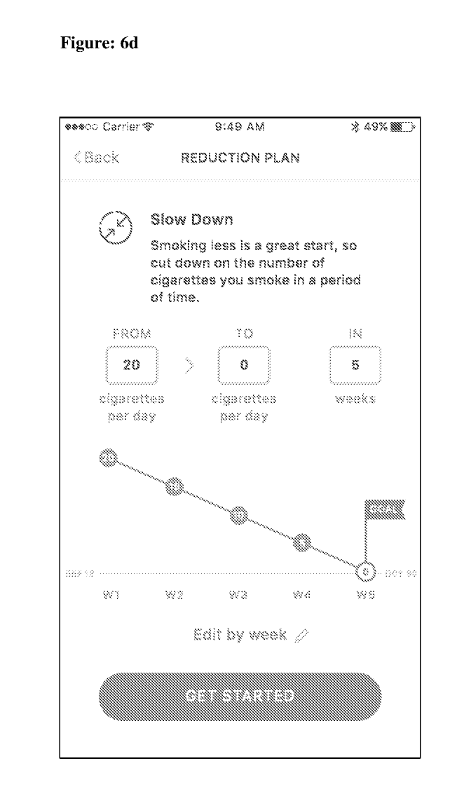

FIGS. 6a-d illustrates the graphical user interface provided on the secondary processing unit which facilitates the monitoring and controlling the usage behaviour of consumption of electronic cigarette. FIG. 6a illustrates the graphical user interface provided on the secondary processing unit which provides the user the options to select one of the approaches to monitor and control the usage consumption of electronic cigarette as described in the earlier section. The user can either choose the “Watch the Road” approach which is same as “Just Track” approach available to the user on the electronic cigarette. Similarly, the “Slow Down” and “Hit the Breaks” approach correlates to the “Reduce” and “Quit Cold Turkey” approaches available to the user on the electronic cigarette.

FIG. 6b illustrates the habit pattern of the consumption of electronic cigarette equivalent to number of conventional tobacco based cigarettes smoked at different locations such as workplace, home etc. by the user, monitored through the graphical user interface on the mobile device (which acts as a secondary processing unit). The electronic cigarette or the device syncs frequently with the mobile device app which maps users' habit pattern to provide them with insight into their habit. The habit pattern is created based on the time of device usage, the amount of usage in a day (number of cigarettes), the location of usage, the trigger for usage and the emotional state during usage.

Every time the device syncs with the mobile device app through the wireless radio transceiver such as Bluetooth, the app is informed about the usage instantly. The app then fetches the location data from GPS provider or Network provider (based on cell tower and Wi-Fi access points), whichever is available. The app populates the detected location data on the interface which user can choose to change manually. The location used might be a precise location or a coarse location. If the user is in certain location radius the app may use geo fencing functionality to tag the location as ‘Home’ ‘Work’ ‘Commute’ etc. The user is then asked to manually choose one of the options for what triggered their urge to use the device and how they are currently feeling.

In scenarios where the device is not in sync with the app for a certain time period, the usage data is stored on the device memory. When the device is later synced with the app, all the data since last sync event is pushed to the app. This data includes date and time tagged usage events. The app while not being in sync with the device, keeps tracking the user location in background. This is achieved in multiple ways which are not battery (resource) intensive. One method of gathering location data is using the ‘passive provider’, which is basically using the location data gathered by other apps/services installed on the user's mobile device. The app will match the date and time of the device usage with available location data and use only the relevant available location data. Another way of capturing the location data in the background is to capture it only when there is a major location change. This lets the app know user's rough location throughout the day which is enough for the app to provide insight to the user about where they smoke the most. Additionally, another method of capturing the location data in the background is to capture the location at particular time intervals of the day. This is done based on the early tracking data where the app knows the usual times of the device usage in a day. User may choose to manually add the trigger and emotion data to all the synced device usage event after the fact.

If the app is not connected, the device stores the information on its memory to be synced with the app next time it connects. The device goes into sleep mode if the user does not continue using the device.

FIG. 6c illustrates the visualization of the cigarettes smoked against the plan created through the graphical user interface on the mobile device (which acts as a secondary processing unit). For the user who wants to follow a regimen, there may be a predefined step-down plan which includes cartridges and daily limits. The user may further track the progress over a period of time against the goal which is preset.

In an event the user sets a goal, the mobile device app starts to track their progress against the goal. For the purpose of simplification, to achieve a set goal, the mobile device app calculates the number of conventional tobacco based cigarettes that the user needs to cut-down on weekly/monthly basis which sets smaller achievable goals. Since the mobile device app tracks the habit, the individual variation is known to the mobile device for creating a personalized plan for e.g. the user might be ‘allowed’ to smoke little more over the weekend and reduce during the week which is in relation to the habit of the user. This flexibility in quitting plan makes it easier for users to follow it through.

FIG. 6d illustrates the plan creation screen for the “Slow Down” approach on the graphical user interface of the mobile device app, which allows the user to set a target or goal date or duration in terms of number of weeks within which the user intends to reduce to “0” number of cigarettes. This target date can be any future date on the calendar and the system uses the daily and/or weekly limits and the target date by which the user intends to cease smoking, to compute, set and display, the number of days, weeks or months the user may be required to quit smoking.

Although the present invention has been described in detail with reference to certain preferred embodiments and examples thereof, other embodiments and equivalents are also possible. Despite the fact that various characteristics and advantages of the present invention have been laid down in the description, but various modifications are still possible in the presently disclosed system and method without deviating from the intended scope and spirit of the present invention.

1. A method of tracking the usage behavior of consumption of an electronic cigarette, the method comprising:
displaying on the body of the electronic cigarette, the amount of nicotine inhaled by the user during the consumption of the electronic cigarette.

2. The method according to claim 1, wherein the amount of nicotine is in the form of percentage of the amount of nicotine from a single conventional tobacco based cigarette.

3. The method according to claim 1, wherein the amount of nicotine is in the form of a number; wherein the number is equivalent to the number of conventional tobacco based cigarettes.

4. The method of controlling the usage behavior of consumption of an electronic cigarette, the method comprising:
enabling the user to manually set a daily consumption limit equivalent to number of conventional tobacco based cigarettes by using the set limit button on the body of the electronic cigarette; and wherein upon reaching the daily consumption limit, the electronic cigarette goes into lock mode where the user cannot use it anymore.

5. The method of claim 4, wherein the electronic cigarette going into the lock mode is notified to the user with a light vibration of the electronic cigarette and display of a ‘X’ symbol on the body of the electronic cigarette.

6. A method of assisting in tracking and controlling the usage behavior of consumption of an electronic cigarette, using one or more secondary processing units, the method comprising:
displaying at least one or more graphical user interfaces inviting the user to enter data describing the user's current smoking habit,
receiving data from the user, the data being indicative of the user's current smoking habit,
displaying at least one or more graphical user interfaces inviting the user to select either a tracking or a controlling approach,
receiving data from the user, the data being indicative of the selected approach.

7. The method of claim 6, wherein the secondary processing unit is a mobile device.

8. The method of claim 7, wherein the graphical user interface is visible on a mobile-device-based application installed on the secondary processing unit.

9. The method of claim 6, wherein the secondary processing unit is a remote personal computer system.

10. The method of claim 6, wherein the data describing a user's current smoking habit includes a list of inquiries to which the user responds using fields for data entry.

11. The method of claim 10, wherein the list of inquiries includes the strength or type of conventional tobacco based cigarette the user would like to consume.

12. The method of claim 6, wherein the data being indicative of the user's current smoking habit includes the strength or type of conventional tobacco based cigarette the user consumes.

13. The method of claim 6, wherein the tracking approach is a Just Track approach.

14. The method of claim 6, wherein the controlling approach is selected from a Quit Cold Turkey approach or a Reduce approach.

15. The method of claim 14, wherein the controlling approach is the Reduce approach.

16. The method of claim 15, wherein the data being indicative of the user's selected approach is used to create a personalized plan for reduction; wherein the personalized plan comprises of tracking of user's smoking habit for the first week, and gradually reducing the number of conventional tobacco cigarettes to be consumed per week in a linear fashion in order to achieve the target date of zero number of cigarettes.

17. A nicotine delivery system comprising:
a) an electronic cigarette comprising:
a mouthpiece (009),
a cartridge (015),
an airflow and vapor sensor (008),
a heating coil (011),
a set limit button (005),
a capacitive sensor (003),
a main power circuit board (002),
a battery (013),
a Light Emitting Diode display (001), and
c) an electronic cigarette holder which holds the electronic cigarette when it is not in use.

18. The nicotine delivery system of claim 17, wherein the cartridge contains nicotine liquid.

19. The nicotine delivery system of claim 17, wherein the cartridge contains non-nicotine liquid.

20. The nicotine delivery system of claim 19, wherein the amount of nicotine in the nicotine liquid is selected from about 0.6 mg, 1.1 mg, 1.6 mg or 2.4 mg.

21. The nicotine delivery system of claim 17, wherein the cartridge further comprises of a chip which acts as a unique Identifier (UID) allowing the primary processing device of the electronic cigarette to detect the type of cartridge (strength of nicotine and/or a non-nicotine cartridge).

22. The nicotine delivery system of claim 17, wherein the electronic cigarette holder comprises of a battery, a charging circuit, an LED indicator and a USB connector.
